(19) United States
(12) Patent Application Publication
(10) Pub. No.: US 2005/0080318 A1
(43) Pub. Date: Apr. 14, 2005

(54) Multi-functional video scope

(76) Inventor: John B. Squicciarini, Aliso Viejo, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/938,688

(22) Filed: Sep. 10, 2004

Related U.S. Application Data
(60) Provisional application No. 60/510,706, filed on Oct. 9, 2003.

(51) Int. Cl. A61B 1/04; A61B 1/303

(52) U.S. Cl. 600/114; 600/129; 600/128; 600/106; 600/109

(57) ABSTRACT

An endoscope has a head with a lens, and an elongated body that includes a shaft and extendable arms. The endoscope further comprises a frame that is configured to extend the arms outward from the body, and a space between the shaft and the arms such that when the arms are extended, the shaft is moveable without necessarily displacing the arms. In preferred aspects, the endoscope has an internal power source and a connector that carries a video signal to an external monitor, and further includes a head that is capped with a lens, behind which a camera and a plurality of light emitters are disposed.

This application claims the benefit of our U.S. Provisional Patent Application with the Ser. No. 60/510,706, filed Oct. 9, 2003, which is incorporated by reference herein.

### FIELD OF THE INVENTION

The field of the invention is endoscopic devices.

### BACKGROUND OF THE INVENTION

There are numerous endoscopic devices known in the art, including for example, a typical endoscope described in U.S. Pat. No. 5,421,339 to Ramanujam et al. In that patent the endoscope comprises a laser with fiber optics carrying light to a probe, and collection fibers carrying induced and reflected light to from the probe to an external sensor. Among other uses described in the '339 reference, the endoscope is used for spectroscopic methods to improve predictive value of colposcopy. While such configurations can have various advantages in stationary use, the light source and image analysis system required for such systems often prevent mobile use.

To render a colposcope more suitable for mobile use, the endoscopic device can be configured to have a head with camera, light emitters, infusion, and suction channels as described in U.S. Pat. App. No. 2002/0022764 to Smith et al., wherein the camera can be located at the end of the probe, or along the side of the probe. Light emitters (or a fiber optic light bundle) can be mounted directly on the end of the head, and a hand held display can be employed in such devices.

Alternatively, as described by Kirsner in U.S. Pat. App. No. 2004/0068162, a device can be configured to enable a patient to perform a colposcopic or other endoscopic self-examination at minimal discomfort, wherein the device transfers the diagnostic information wirelessly to a medical professional. Such endoscopic devices are particularly suitable where frequent self-examination in a private environment is desired. Unfortunately, Kirsner's device fails to provide any test results to a patient, and only advises the patient whether to see the physician or not. Moreover, implementation of Kirsner's device to detect early signs of cervical cancer is hindered by the fact that the sensor appears to be on side of the shaft. Such sensor position typically prohibits generation of a perspective view as the sensor is positioned adjacent the tissue being examined.

Thus, while numerous compositions and methods for endoscopic devices are known in the art, all or almost all of them, suffer from one or more disadvantages. Therefore, there is still a need for improved endoscopic devices.

### SUMMARY OF THE INVENTION

The present invention is directed to configurations and methods for a visualization device having a head with a lens. Contemplated devices further include an elongated body having a shaft and extendable arms, and a frame that extends the arms outward from the body. A space is configured between the shaft and the arms such that when the arms are extended, the shaft is moveable without necessarily displacing the arms.

In especially preferred aspects, a lens is mounted on the head, and a camera and a light emitter can be disposed in the head and/or the body. Especially preferred heads have a base with a substantially round outer boundary, wherein the extendable arms are configured to retract to provide a substantially continuous outer boundary with the base. While not limiting to the inventive subject matter, the extendable arms can comprise a first, a second, and a third arm.

Additionally or alternatively, the frame extends the arms outward from a retracted position about the body to an extended position farther displaced from the shaft, and the device can further include a working tool (e.g., Pap smear collector, an ultrasound emitter, or a fluid line for a cryogenic fluid, dye, or lavage fluid) deployable from a storage position that is at least partially in a location between the shaft and at least one of the arms. Contemplated devices will further include a connecter that carries a signal from the camera to a monitor.

In one aspect of the inventive subject matter, a visualization probe includes a head capped with a lens, behind which are functionally mounted a camera and a plurality of light emitters, an internal power source, and a connector that carries a video signal to an external monitor. In particularly preferred devices, the light emitters comprise at least three diodes disposed about an aperture of the camera, most preferably wherein at least two of the light emitters produce light of significantly different colors from one another. Thus, in most preferred devices, it should be recognized that the light passes to the camera without use of fiber optics. In still further contemplated aspects, the connector includes a radio transmitter.

Additionally, or alternatively, contemplated devices include a shaft, and at least one tool (e.g., Pap smear collector, fluid line, or an ultrasound emitter) is disposed in a storage position adjacent the shaft. An elongated stabilizing arm can be moveably disposed between a retracted position close to the shaft and a deployed position wherein at least part of the arm is distanced from the shaft.

Various objects, features, aspects and advantages of the present invention will become more apparent from the accompanying drawing along with the following detailed description of preferred embodiments of the invention.

### BRIEF DESCRIPTION OF THE DRAWING

FIG. 1 is a schematic vertical cross sectional view of an exemplary device in collapsed configuration according to the inventive subject matter.

FIG. 2 is a schematic view of the device of FIG. 1 in extended configuration.

FIG. 3 is a schematic view of the device of FIG. 2 with the head in retracted position.

FIG. 4 is a schematic view of the device of FIG. 3 in which an exemplary tool is extended through the head.

FIG. 5 is another schematic view of the device of FIG. 1 in use as a colposcopic device.

FIG. 6 is another schematic view of the device of FIG. 2 in use as a colposcopic device.

FIG. 7 is another schematic view of the device of FIG. 3 in use as a colposcopic device.

FIG. 8 is another schematic view of the device of FIG. 4 in use as a colposcopic device.

### DETAILED DESCRIPTION

The inventor has discovered that an endoscopic device can be configured to allow use of the device in situ in multiple configurations, and especially to allow examination of a tissue using a plurality of perspective positions of the optical head relative to the examined tissue while maintaining the device in substantially the same position. Contemplated devices further advantageously provide a mechanism that temporarily displaces tissue from the optical field that would otherwise obstruct optical examination using a device without that mechanism.

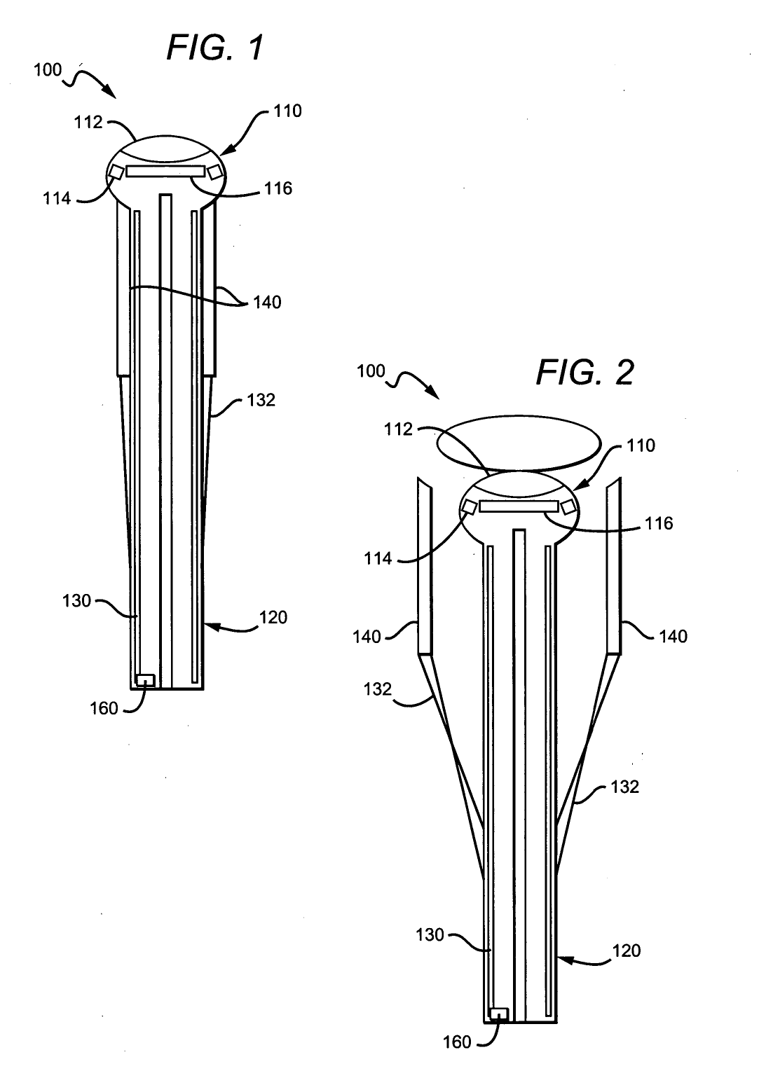

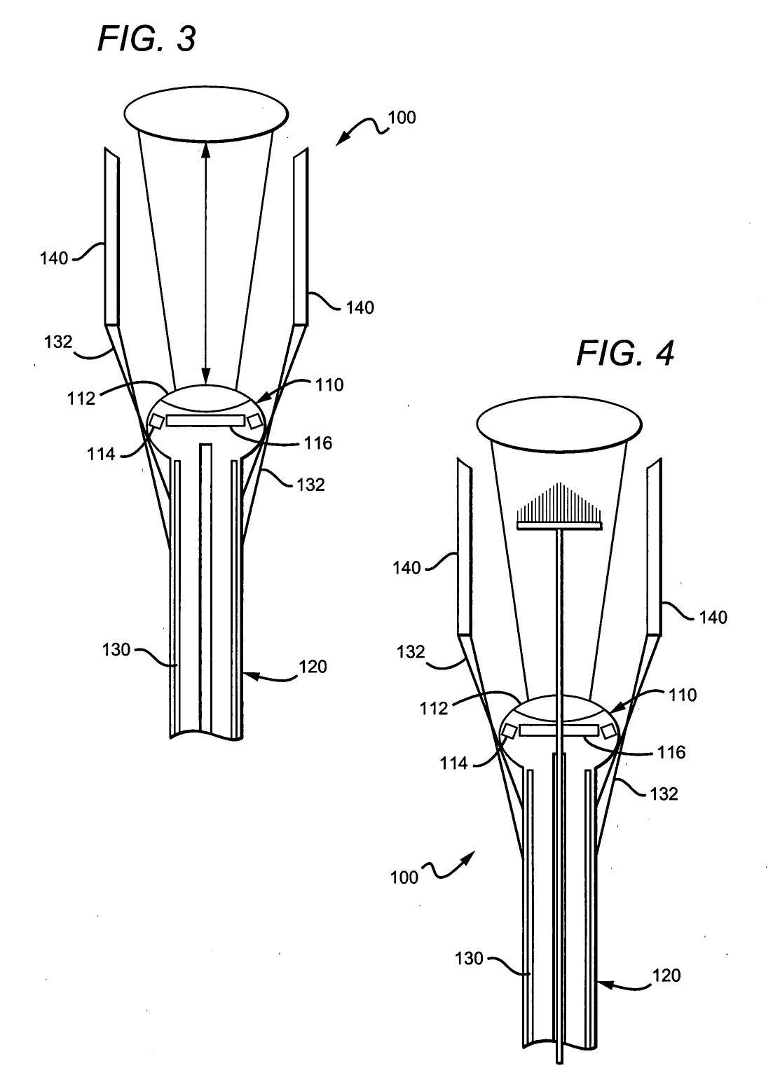

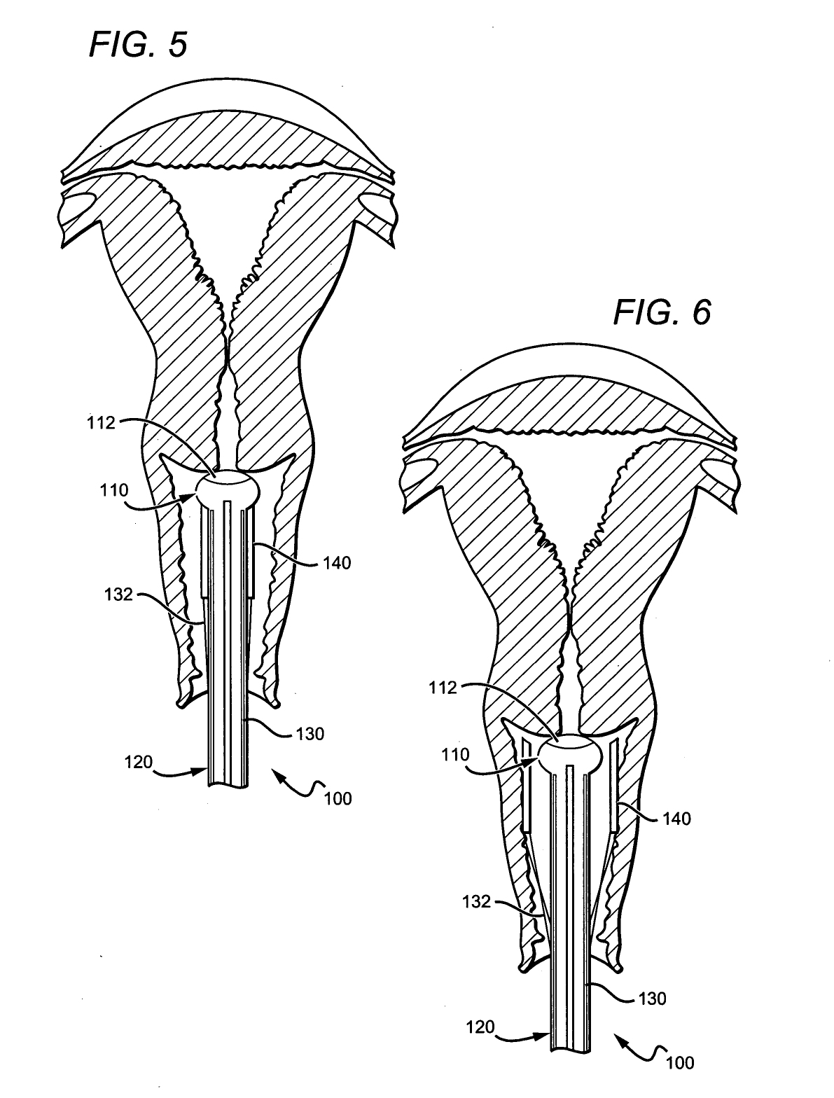

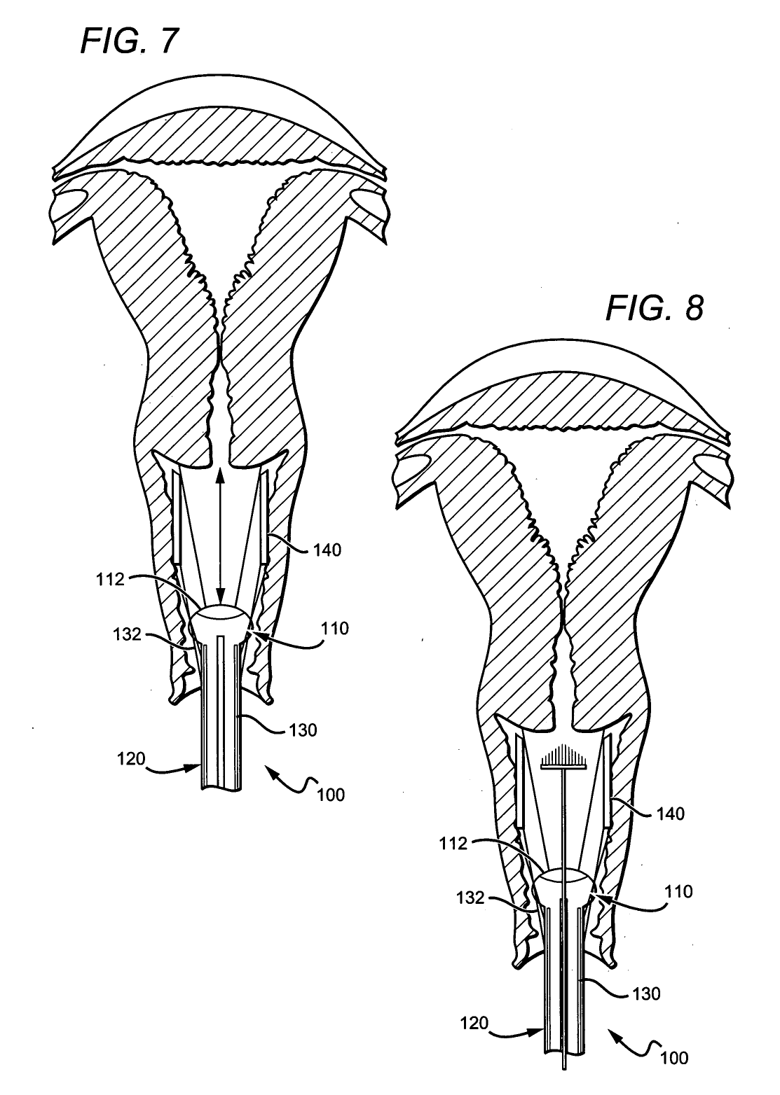

An exemplary endoscopic device 100 in a first configuration (collapsed) is depicted in FIG. 1 in which a head 110 having lens 112 is coupled to a shaft 120. Light source 114 and camera 116 are further disposed within the head 110, but may also be located outside of the device where the device is disposable and coupled to a hand-held base (the light and/or image is transferred through the device to the base via one or more light guides). Coupled to the shaft 120 is frame 130 (having tracks as indicated), wherein extendable arms 140 (only two shown) are coupled to the frame 130 via connectors 132 that move within the tracks. Working tools (not shown, see FIG. 4) are disposed within a space formed between the shaft and the arms, and connector 160 provides video signals from the camera 116 to an imaging device. Alternatively, where the imaging device is disposed in a hand-held base, the connector 160 can be omitted (not shown). FIG. 2 depicts the device of FIG. 1 in a second (extended) configuration in which the head is proximal to a target tissue, while in FIG. 3 the head is moved in a distal position relative to the target tissue without moving the endoscopic device in situ (without repositioning the arms relative to the adjacent tissue). It should be noted that using such configuration, the viewing angle and/or field can be continuously changed without changing the position of the endoscopic device in situ. FIG. 4 depicts deployment of a tool from the device. FIGS. 5-8 depict the device of FIGS. 1-4, respectively, in use as a colposcopic device. With respect to the elements in FIGS. 2-8, the like numerals depict like elements as depicted in FIG. 1.

In an especially preferred aspect of the inventive subject matter, contemplated devices will include a head with a lens, and an elongated body comprising a shaft and one or more extendable arms. In such devices, a frame is coupled to at least one of the arms and configured to extend the arm or arms outward from the body, wherein a space is provided between the shaft and the arm or arms such that when the arms are extended, the shaft is moveable without necessarily displacing the arms.

Depending on the particular use of the endoscopic device, it should be recognized that the head can have various sizes and configurations. However, it is generally preferred that the head is round, and more preferably at least partially ovoid or spheroid. Viewed from another perspective, the head can be advantageously configured such that the device is advanced to the target tissue without injury to adjacent tissue. Furthermore, the shape of the head is at least partially depending on the lens and/or optical system that is preferably disposed in the head. Most typically, the head will have a diameter of less than 2 inch, more typically of less than 1 inch, and most typically of less than ½ of an inch. In preferred configurations, the head has a portion that engages with at least one arm to retain the arm in a retracted position (typically where the arm has a minimum distance to the shaft), wherein the retention can be achieved using a movable retention member, or simply by engaging the arm in a corresponding indentation or other non-movable structure. Thus, contemplated heads will include those having a base with a substantially round outer boundary, wherein the extendable arms retract to provide a substantially continuous outer boundary with the base.

In further preferred aspects, the head is continuous with the shaft and coupled to the shaft in a removable manner. While not limiting to the inventive subject matter, the shaft has a diameter that is preferably less than that of the head to accommodate (among other elements) the frame and the arms. Depending on the use of the endoscopic device, the shaft can have a diameter of less than 1 inch, more typically of less than ¾ of an inch, and most typically of less than ½ of an inch, while the length of the shaft can be between 2 inches and 20 inches, and even longer. Similarly, the shaft can be configured to be flexible (e.g., passively via deformation, or actively via guide mechanism) where the endoscope is used as a colonoscope, or rigid where the endoscope is used as a laparoscope or colposcope.

Most preferably, the head of contemplated devices will include a lens that can further include additional optical components to allow focusing, zooming, and/or change of an angle of visual inspection relative to a hypothetical axis that is parallel to the shaft. For example, and depending on the particular use of the endoscope, the lens can be an integral part of the head in some devices, while in other devices the lens or optical unit is removably placed into the head. With respect to a particular focusing arrangement, it should be recognized that the lens and/or optical system can have a fixed focus (and with that a fixed magnification) or a variable focus and variable magnification. There are numerous such optical devices and configurations known in the art, and all of such devices and configurations are deemed suitable for use herein.

Where the focus is fixed, it is especially preferred that the tip can contact the tissue to be examined to thereby provide the highest magnification. Alternatively, the focal depth may be larger, for example at least ¼ inch, more preferably at least ½ inch, and most preferably 1-20 inches. In such devices, and depending on the particular use, the preferred fixed magnification is between 1× (or less) to about 10× (or higher). On the other hand, where the focus can be adjusted, focal depth can be less than ¼ inch, and more typically less than 1/8 inch. In such devices, and depending on the particular use, the preferred fixed magnification is between 133 (or less) to about 20× (or higher). Furthermore, it should be recognized that contemplated devices can also include a second lens and/or optical system to allow stereoscopic imaging. For the second lens and/or optical system, the same considerations as provided above apply. Additionally, it is contemplated that the lens and/or optical system can include one or more filters (e.g., to filter out excitation light reflected from a tissue, or to provide a narrow band of excitation light).

Most preferably, the lens and/or optical system is further configured such that the viewing angle can be continuously changed from a coaxial to an angled position. Such configuration is particularly advantageous where the target tissue is not directly in front of the lens/optical system, but offset to one side where a conventional video scope would not be able to acquire an image without altering its configuration (e.g., by bending the tip). For example, suitable angles will include those between 0 degrees and 60 degrees (and even more), more preferably between 0 degrees and 45 degrees, and most preferably between 0 degrees and 30 degrees.

It is generally contemplated that the head and/or body may include a camera and a light emitter. There are numerous cameras suitable for endoscopic use known in the art, and all of them are contemplated herein. Especially preferred cameras include a video chip that not only registers light of a wavelength between about 400 nm to about 720 run, but also near-UV (between 350 nm and 400 nm), UV (less than 350 run), near IR (between 720 nm and 780 nm), and IR (more than 780 run) light. Suitable video chips can provide the data directly to a connecter that carries the video signal from the camera to a computer and/or monitor, or can provide the data to a processor that is also disposed in the shaft or head. Data transfer to the processor, computer, and/or monitor can be conventional (e.g., using USB port) or wireless (e.g., using Bluetooth™ technology).

Alternatively, in an especially preferred aspect of the inventive subject matter, at least one of the camera and the light source is disposed in a device (most preferably hand-held) to which the body and head are removably coupled. In such devices, it should be appreciated that the curvature of the lens is parallel to, or even part of the head, and that the lens is optically coupled to the camera via a fiber optic arrangement (or other light guide). Viewed from another perspective, the lens may be an integral part of the head and the light collected by the lens is transferred to the camera (which is disposed in the device that is removably coupled to the body) via fiber optic. Preferably, the light emitter in such devices is also disposed in the device that is removably coupled to the body, and the light is provided to the head via a plurality of optic fibers or other light guides. Most preferably, the light is delivered to the area to be examined in a homogenous manner to allow visualization of an evenly illuminated field.

Thus, it should be recognized that the head and body (together with the frame and optional tools) may be configured as a detachable unit (and most preferably as a unit that is disposed after single use) that can be removed from a device that includes the camera and/or light source. In such configurations, the arms are preferably actuated using a mechanism that is coupled to the body but not to the device that includes the camera and/or light emitter. Exemplary suitable configurations for devices that include the camera and/or light emitter are described in our co-pending provisional application with the title “Flexible Video Scope Extension and Methods”, which was filed concurrently with the present application, and which is incorporated by reference herein.

With respect to the arm or arms of the device, it is generally preferred that the device includes a plurality of arms that are coupled to the shaft such that the arms move between a first position and a second position, wherein the arms are proximal to the shaft in the first position and distal to the shaft in the second position. There are numerous manners possible in which the arms can be moved from the first to the second position, and all known manners are deemed suitable for use herein. For example, the arms can be moved by a plurality of actuators that are coupled to the shaft, wherein the actuators are moved by one or more elements (e.g., hydraulic, mechanic, or electric) at least partially disposed in the shaft. More preferably, however, the arms are moved by a frame that is coupled to the outside of the shaft, wherein the frame comprises a plurality of actuators (e.g., using umbrella type actuation) that will move the arms from the first to the second position. There are numerous manners of such actuation known in the art, all of which are deemed suitable for use herein. For example, the frame can have a set of guide rails that extend longitudinally along the shaft, wherein the frame is coupled to the shaft. One end of a first actuator can slidably engage with the guide rail while the other end can engage with one portion of an arm. A second actuator can move the first actuator along the guide rail while a third actuator engages with the second actuator to provide additional moving force. Alternatively, a plurality of actuators can be threaded through the shaft and perpendicularly exit the shaft to connect with the arms or arms. Advancing the actuators in such devices into the shaft will move the arm or arms outwardly. Most preferably, the endoscopic device has at least three arms that are circumferentially disposed on the shaft, and that are coupled to the shaft via a frame. The frame preferably extends the arms outward from a retracted position about the body to an extended position farther displaced from the shaft. In still further alternative aspects, the arms may also be moved via a screw-motion.

Depending on the particular configuration of the frame, shaft, and/or arms, it is contemplated that the endoscopic device further includes a working tool deployable from a storage position that is at least partially defined by a space between the shaft and at least one of the arms. For example, where the endoscopic device is a colposcopic device, suitable working tools include a pap smear collector, a fluid line, and/or an ultrasound emitter. Further contemplated devices can also have one or more fluid lines that carry a cryogenic fluid, a dye, and/or a lavage fluid. In another example, where the endoscopic device is a colonoscope, suitable working tools can include a deployable scissor, a cauterizing loop, a needle for injection of pharmaceutical compositions, etc.

In another aspect of the inventive subject matter, contemplated devices can also include a head that is capped with a lens, behind which are functionally mounted a camera and a plurality of light emitters. An internal power source can provide power to the camera and/or the light emitters, and a connector carries a video signal to an external monitor (with respect to the configuration of the head and coupling to the shaft, the same considerations as provided above apply).

Preferably, the light emitters comprise at least three diodes disposed about an aperture of the camera, wherein at least two (and more preferably three) of the light emitters produce light of significantly different colors from one another (i.e., with wavelength maximum at least 20 nm apart). Alternatively, light sources other than a light diode can be used, and suitable alternative light sources include incandescent, laser, and electro-luminescent light sources. Depending on the particular requirements, the light source can be disposed behind a filter that modifies the spectral characteristics of the light source, or can be transmitted via a light guide. However, it is particularly preferred that the light from the light source passes to the camera without use of fiber optics or other light guides.

As the camera in such devices is located in the head, it is contemplated that supporting electronic devices can be positioned in the shaft, and more preferably outside the endoscopic device. Consequently, it should be recognized that such devices will include an interface that transfers the image data (processed or raw) to an imaging device. Preferably, such interface will comprise a connector, and most preferably a wireless connector (e.g., using a radio transmitter).

It is still further contemplated that the devices in which the head includes the camera and a plurality of light emitters have a shaft (preferably as described above), and that at least one tool is disposed in a storage position adjacent the shaft. Furthermore, it is also preferred that the shaft is coupled to a frame and one or more elongated stabilizing arms that are moveable between a retracted position close to the shaft, and a deployed position distal to the shaft. With respect to the various frame configurations that provide movability of the arms to extend outwardly from the body and the movability of the head relative to the target tissue while the device is in situ, the same considerations as discussed above apply.

Thus, specific embodiments and applications of multi-functional video scopes have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps can be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. A visualization device comprising:
a head with a lens;
an elongated body including a shaft and extendable arms;
a frame that extends the arms outward from the body; and
a space between the shaft and the arms such that when the arms are extended, the shaft is moveable without necessarily displacing the arms.

2. The device of claim 1, wherein the lens comprises a fixed focus lens.

3. The device of claim 1, further comprising both a camera and a light emitter disposed in at least one of the head and the body.

4. The device of claim 1, wherein the head has a base with a substantially round outer boundary.

5. The device of claim 1, wherein the head has a base, and the extendable arms retract to provide a substantially continuous outer boundary with the base.

6. The device of claim 1, wherein the extendable arms comprise a first, a second, and a third arm.

7. The device of claim 1, wherein the frame extends the arms outward from a retracted position about the body to an extended position farther displaced from the shaft.

8. The device of claim 1, further comprising a working tool deployable from a storage position at least partially defined by a space between the shaft and at least one of the arms.

9. The device of claim 1, wherein the working tool comprises at least one of a Pap smear collector, a fluid line, and an ultrasound emitter.

10. The device of claim 9, wherein the fluid line carries at least one of a cryogenic fluid, a dye, and a lavage fluid.

11. The device of claim 1, further comprising a connecter that carries a signal from the camera to a monitor.

12. A visualization probe comprising:
a head capped with a lens, behind which are functionally mounted a camera and a plurality of light emitters;
an internal power source; and
a connector that carries a video signal to an external monitor.

13. The probe of claim 12, wherein the light emitters comprise at least three diodes disposed about an aperture of the camera.

14. The probe of claim 12, wherein at least two of the light emitters produce light of significantly different colors from one another.

15. The probe of claim 12, wherein at least three of the light emitters produce light of significantly different colors from one another.

16. The probe of claim 12, wherein light passes to the camera without use of fiber optics.

17. The probe of claim 12, wherein the connector includes a radio transmitter.

18. The probe of claim 12, further comprising at least one tool selected from the group consisting of a Pap smear collector, a fluid line, and an ultrasound emitter.

19. The probe of claim 12, wherein the probe has a shaft, and the at least one tool is disposed in a storage position adjacent the shaft.

20. The probe of claim 12, wherein the probe has a shaft, and further comprising at least one elongated stabilizing arm that is moveable between a retracted position close to the shaft, and a deployed position wherein at least part of the arm is distanced from the shaft.
